(19) United States
(12) Patent Application Publication
(10) Pub. No.: US 2022/0192815 A1
(43) Pub. Date: Jun. 23, 2022

(54) ENDOVASCULAR PROSTHESIS

(71) Applicants: MDB TEXINOV, Saint Didier DeLa Tour (FR); DELTAVAL, Lyon (FR)

(72) Inventors: Jacques Tankere, Meximieux (FR); Eric Mignot, Velanne (FR); Franck Ott, Voiron (FR)

(21) Appl. No.: 17/432,519

(22) PCT Filed: Mar. 5, 2020
(86) PCT No.: PCT/FR2020/050447

§ 371 Date: Aug. 20, 2021

(30) Foreign Application Priority Data
Mar. 18, 2019 (FR) 1902757

(51) Int. Cl. A61F 2/07; D04B 21/20; A61L 31/06; A61L 31/02; A61L 31/10; A61L 31/14; D04B 21/16

(52) U.S. Cl. CPC A61F 2/07; D10B 2331/04; A61L 31/06; A61L 31/022; A61L 31/10; A61L 31/14; D04B 21/16; A61F 2210/0014; A61F 2230/0069; A61F 2230/0091; A61F 2240/001; A61L 2400/16; A61L 2400/18; D10B 2509/06; D04B 21/205

(57) ABSTRACT

This vascular stent graft (1) is formed of a tubular knitted textile structure, integrating within the meshes of said knitted textile structure at least one helical continuous weft yarn (3) extending all along the major dimension of the stent graft.

Said at least one stent graft (3) is made of a shape memory alloy having previously been submitted to such an education, and in particular such a thermomechanical treatment that, at the human body temperature, said yarn gives the structure its tubular shape by superelasticity or shape memory effect.

### TECHNOLOGICAL FIELD

The invention pertains to the field of vascular endoprostheses or stent grafts, more particularly intended to be implemented in the case of aortic aneurysms.

The invention more particularly aims at such vascular stent grafts integrating a tubular textile structure and at least one filamentary element of shape memory alloy type.

### BACKGROUND

Aneurysm is a dilatation of an arterial section. The rupture of this pathological area is likely to cause hemorrhage, sometimes fatal. When such an aneurysm is detected, two solutions are likely to be implemented by the medical profession: the first one comprises resorbing this aneurysm by open surgery, that is, by a particularly heavy and invasive technique. The second one comprises shunting said aneurysm by inserting at its level a stent graft, implanted within the considered artery upstream and downstream of said aneurysm; it is then spoken of endovascular surgery. This shunt is intended to allow the circulation of the blood flow within the stent graft and, as a corollary, induces the decrease of the blood pressure within said aneurysm and, accordingly, the risk of rupture thereof. The advantage of this second solution mainly is to significantly reduce the actual operation.

Such stent grafts are conventionally formed of a textile body associated with an exoskeleton, typically made of steel or of a metal alloy and advantageously having superelastic or shape memory properties. This exoskeleton is usually sutured onto the body made of textile material or associated with the stent graft during an operation called electrospinning in the considered field. The body made of textile material is for example made of PET (polyethylene terephthalate).

Shape memory materials are now widely known and used. They have the characteristic of being capable of reversibly reaching relatively significant deformation levels. This phenomenon is due to a crystallographic phase transformation within the material when the latter is submitted to a mechanical loading—to stress—and/or to a temperature variation. Thus, shape memory materials are capable of developing several particularly advantageous properties, such as superelasticity and ferroelasticity (shape memory). Among shape memory alloys, those based on nickel and titanium, such as for example those known under trade name Nitinol, are particularly known.

There has thus been described, for example, in document U.S. Pat. No. 6,814,747, such a stent graft comprising a textile tubular sheath, to the outside of which are more or less periodically added rings made of a shape memory alloy, likely to expand under the effect of temperature, and in the case in point typically under the effect of the human body temperature, in such a way as to give the stent graft the tubular shape required to ensure its shunt function and, as a corollary, to give the stent graft a sufficiently small shape to allow its introduction into the considered artery, for example, by means of a catheter.

While, undoubtedly, this type of stent graft has allowed a significant progress in the treatment of aneurysm by endovascular surgery, it however as a number of disadvantages.

Among these, one may first mention the case of endoleaks, that is, a lack of tightness of the installed stent graft. These endoleaks are inherent to the actual structure of the tubular sheath, typically formed by weaving, in addition to the suture areas of the memory shape alloy exoskeleton on said sheath, likely to create weak areas at the level of the tubular sheath, and thus as a corollary of the orifices through which the blood flow is likely to escape. While, undoubtedly, this type of stent graft has allowed a significant progress in the treatment of aneurysm by endovascular surgery, it however as a number of disadvantages. When such a stent graft is for example implanted at the level of the aorta, particularly abdominal, and when the pressure of the blood flow at this level is known, these leaks may rapidly become a problem, and in particular not allow the resorption of the aneurysm that the stent graft is precisely designed to reduce.

Another difficulty inherent to the use of prior art stent grafts lies in mispositioning and an unsatisfactory behavior at the level of the plications, that is, of the tortuosities that the stent graft is likely to take in order to adapt to the specific meanders of the artery at the level of which the stent graft is intended to be implanted. Such plications of the stent graft are likely to result in an at least partial occlusion thereof, resulting in significant complications requiring a new surgical operation.

Still another difficulty resulting from prior art stent grafts lies in their heterogeneity. Indeed, due to the suture or to the ring fastening mode or the like system, and generally to the exoskeleton on the textile structure, the stent graft has a mechanical behavior which deviates from that of the native tissues, resulting in more or less rapid rejections of the stent graft, requiring their periodic replacing due to new risks of occlusion likely to occur and not to result in the desired effect, in the case in point the resorption of the aneurysm. Further, the heterogeneities of prior art stent graft structures may result in a potential slipping of said structure within the artery—migration—, this inevitably resulting in the need for a second highly invasive operation.

### SUMMARY OF THE DISCLOSURE

The invention aims at an endovascular stent graft aiming at overcoming these different disadvantages.

It thus aims at an endovascular stent graft formed of a tubular knitted textile structure, integrating within the meshes of said knitted textile structure at least one helical continuous weft yarn, extending all along the major dimension of the stent graft, said at least one yarn made of a shape memory alloy having previously been submitted to such an education, and particularly a such thermomechanical treatment, as to gives it superelastic properties or during the austenitic transformation resulting from the human body temperature, so that said yarn gives the structure its tubular shape.

In other words, the invention comprises suppressing any form of exoskeleton and of possible sutures thereof onto the textile structure of prior art, providing the stent graft an optimal homogeneity, and developing, due to the optimization of the stitches of the knitted textile structure and to the thermomechanical behavior of the weft, as successful a biomimetic behavior as possible.

Due to the integration within its structure, and more precisely within the meshes of the knitted textile structure, of a continuous shape memory alloy yarn, the latter thus provides, once the stent graft is in plane, the desired tubular shape, to allow it to fulfill its primary function, that is, allowing the circulation of the blood flow therethough.

According to the invention, the knitted textile structure is obtained on a double-needle bed Rachel loom or on a hook loom, according to a Charmeuse weave or other similar bindings of double knit, weft chain type, or any other weave enabling to obtain good biomimicry characteristics of the implanted structure, and according to the desired mechanical characteristics, particularly elasticity, extensibility, or another behavior.

It should further be specified that due to the electronic controls of the implemented knitting machines, it is possible to change weave during the manufacturing, and for example to have areas of different density or characteristics.

The textile structure in question is made based on PET (polyethylene terephthalate) or of any other polymer yarn which is biocompatible (polypropylene, ePTFE—expanded polytetrafluoroethylene, for example) and/or resorbable or biodegradable (for example, of PGA—polyglycolic acid type).

The weft yarn, made of a shape memory material and advantageously of Ni—Ti, is continuously inserted into the meshes of said knitted textile structure, the insertion being performed at the level of the double needle bed with an offset of this insertion in the production direction, this continuous weft being conveyed to the level of the two needle beds by revolution around the latter, and thus concurrently with the forming of the two textile layers resulting from the knitting at the level of the two needle beds. The aim of the Ni—Ti weave is first to ensure the main functions of the stent graft, and in particular an optimal deployment of the structure within the aneurysm during the endovascular operation surgery.

This may thus result in a helical arrangement of the weft yarn. The metal skeleton is thus directly continuously integrated within the textile structure, particularly homogeneously and with no suture, conversely to prior art stent grafts, which have exoskeletons generally sutured onto the textile, then resulting in heterogeneous structures.

According to the invention, the weft yarn typically made of a nickel-titanium alloy may itself be submitted to a prior corrugation before its insertion within the double needle bed. This specific corrugation of the weft yarn, having its amplitude and its pitch controlled and imposed during the specific thermal treatment of said yarn, enables to provide more flexibility to the structure and to help the final crimping phase necessary to enable the insertion of said structure into its catheter for its in situ implantation.

The weft yarn made of a nickel-titanium alloy has a diameter in the range from 50 to 200 micrometers. If, indeed, the yarn diameter is smaller than 50 micrometers, experience proves that the primary function provided to the yarn, that is, to give the stent graft its tubular shape after the austenitic transformation, is insufficient, and risks of occlusion are likely to appear.

If, however, the diameter of the yarn forming the weft is greater than 200 micrometers, the stent graft becomes too stiff and its behavior diverges too significantly from the desired biomimicry, likely to result in a risk of migration of the stent graft within the artery into which it is introduced and, as a corollary, to the risks of leaks.

According to another feature of the invention, the longitudinal deformation of the stent graft, that is, along its major dimension, is in the range from 0 to 30%. This deformation may turn out being necessary to remain close to a biomimetic behavior and thus to an optimum and homogeneous flow, decreasing risks of disturbances on other areas of the arteries. It is inherent to the only knitted textile structure that enables such resistance-elongation adjustments.

According to an advantageous feature of the invention, the stent graft once formed is submitted to a compaction of its walls by a mechanical action, and then coated with a specific surface coating (mainly formed of collagen, of albumin, or of gelatin, this coating solution being advantageously completed with components necessary to the biocompatibility of the structure, such as heparin, carbon, or fluoropolymers), to give said stent graft the permeability level required regarding the viscosity of the blood flow, and thus as a corollary to avoid leaks, and typically a permeability close to 0.1 ml/cm²/min determined according to the ISO 25539-2 standard.

The invention also aims at a method of forming this endovascular stent graft. The method comprises:

forming by warp stitch knitting on a double needle bed loom two layers joined together at the level of their respective edges in the production direction to eventually define a tubular structure;

and inserting a continuous weft made of shape memory yarn at the level of the double needle bed, inserting into the meshes on said two layers with an offset in the production direction, the continuous weft being conveyed to the level of the needle beds by revolution around said needle beds, typically helically with respect to the production direction of the structure.

According to an advantageous feature of the invention, the pitch of the helix is constant and, to obtain the required compactness and optimize the tightness of the stent graft, this pitch is 1/1, that is, the weft yarn made of a shape memory material turns around the textile structure once for every stitch. However, according to the desired characteristics, this pitch may be from ½ to 1/10.

### BRIEF DESCRIPTION OF THE DRAWINGS

The foregoing and other features and advantages of the present invention will be discussed in detail in the following non-limiting description, in connection with the accompanying drawings, in which:

FIG. 1 is a simplified view illustrating a stent graft according to the invention implanted within the abdominal aorta.

FIG. 2 is a simplified view of two types of stent graft according to the invention, respectively illustrating a constant-diameter stent graft (left-hand portion) and a stent graft provided with a limb obtained directly during the manufacturing (right-hand portion), and the two types of stent graft may be assembled during the implantation on the patient.

FIG. 3 is a simplified view of a stent graft according to the invention in its phase of forming on a double needle bed loom, showing the weft yarn with superelastic properties.

FIG. 4 is a simplified representation of a detail of FIG. 3.

FIG. 5 is a simplified representation in top view of the principle implemented by the method of forming the stent graft of the invention.

FIG. 6 is a simplified representation in top view of a double needle bed with a representation of the insertion of the shape memory weft yarn looping at the level of said needle beds.

FIG. 7 is a simplified perspective representation of a device capable of implementing the method of the invention.

### DETAILED DESCRIPTION

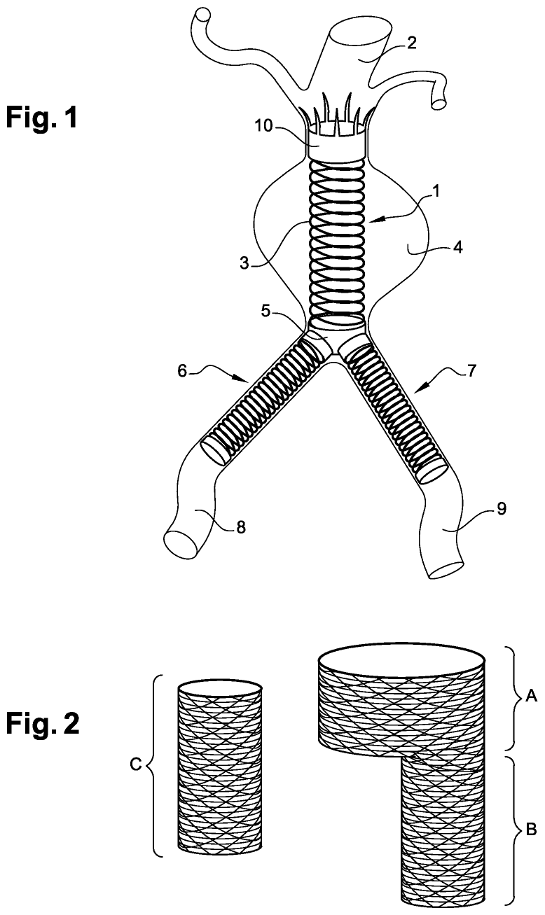

There has been schematically described in relation with FIG. 1 the implantation of a stent graft (1) according to the invention within the abdominal aorta (2). For the simplicity of the illustration, only the continuous weft yarn (3) made of a shape memory alloy, and in the case in point a nickel-titanium alloy, such as for example, of Nitinol, has been shown. Its helical travel, resulting from the embodiment of said stent, can be well observed and, is besides, described in further detail hereafter.

In this example, the main stent graft, intended to shunt the aneurysm (4), splits at the level of a junction area (5) into two secondary stent grafts (6, 7), intended to be implanted in the beginning of the two femoral arteries (8, 9).

The upper area of the stent graft is attached at the level of the aortic arch. In the case in point, experience proves that the area of attachment (10) to the aortic neck should be reinforced with respect to the body of the structure to avoid any risk of migration once the structure has been implanted in vivo. The nickel-titanium weft yarn may thus, in said area, have a different diameter or thermomechanical behavior to ensure the holding of the stent graft.

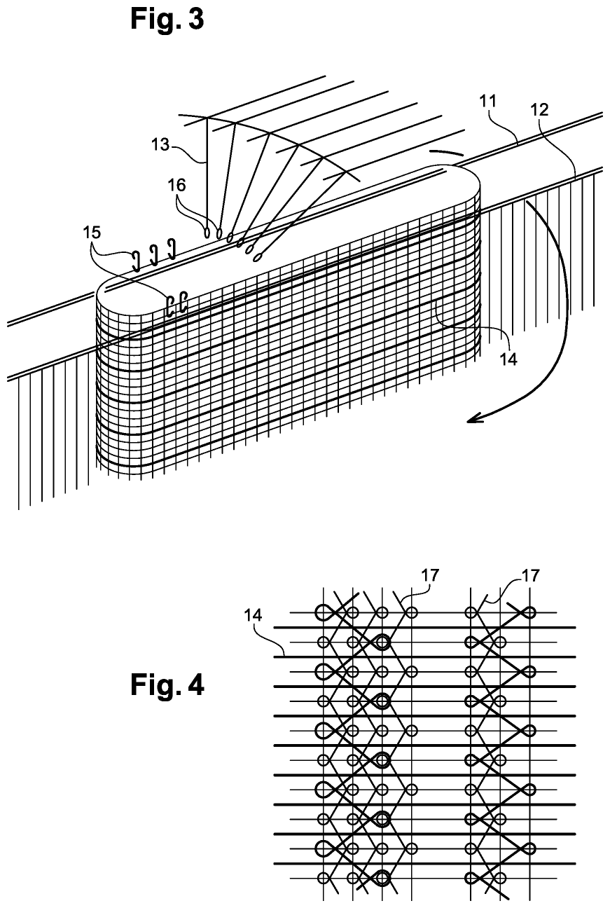

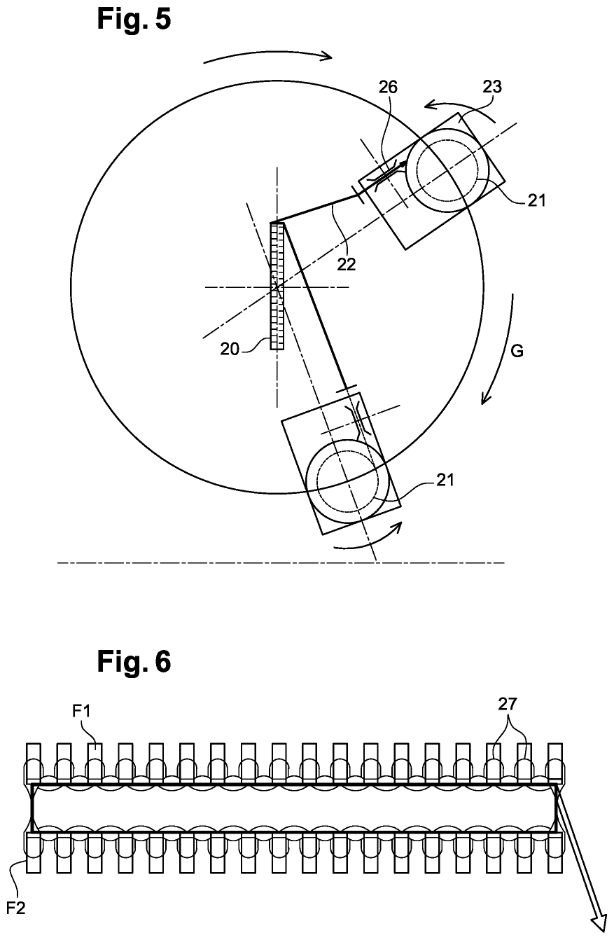

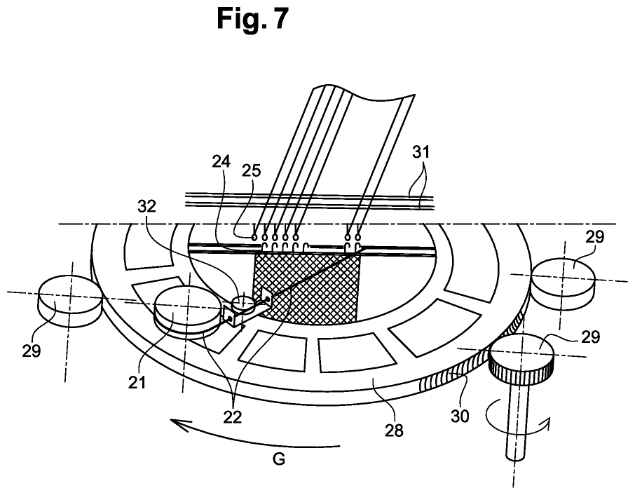

FIG. 3 schematically shows the forming of the stent graft according to the invention on the two needle beds (11, 12) of a Rachel loom with the meshing yarn bars (13) and the continuous nickel-titanium alloy weft yarn (14). The latter alternately passes on each of the two needle beds while integrating into the meshes on each side (needles (15) and guides (16)) by a rotating device (see FIGS. 5 and 7), with a pitch of 1/1 or from ½ to 1/10 by stopping of said rotating device. The rotation of the weft yarn (14) around the two needle beds has been illustrated with an arrow.

FIG. 4 shows a detail of FIG. 3. More precisely, the meshing within the needle beds has been schematically shown. The yarns (17) forming the structure and for example made of PET meshing around the weft yarn (14) thus appear.

This embodiment further enables to decrease by programming the width of the tube eventually defined by the 3D textile structure during the manufacturing cycle to form one of the limbs or secondary stent grafts (6, 7) of an aortic aneurysm prosthesis, intended to insert into one or the two femoral arteries (see FIG. 2).

According to this specific embodiment (FIG. 2, right-hand portion), the second limb (6, 7) is separately formed on the knitting machine, according to a diameter smaller than that corresponding to the main stent graft. The assembly is formed during the implantation by interlocking and attaching according to a mode respecting the requirements of stent grafts.

The junction area (5) between the different portions (3, 6 and 7) is formed by knitting, by weaving or any other assembly means, according to a geometry specific to the desired configuration, and enables to continuously bind the entire structure.

This secondary stent graft is introduced in situ by means of a second catheter as usually done for stent grafts. In this case, during the implantation, the narrowest portion hooks into the first portion at the coming out of the catheter. The limb diameter is slightly greater than that of the portion that receives it, which enables it to exert an effort on the first portion, sufficient to guarantee its maintaining in vivo. According to needs, additional hooks may be added to exclude any risk of sliding or of separation of the different portions.

The implantation of this stent graft in situ is performed by means of one or a plurality of catheters, having said stent graft inserted therein. This insertion is generally consecutive to a step of crimping of the 3D structure forming said stent graft. Once the latter is in place, in the case in point within the abdominal aorta, the superelasticity of the nickel-titanium weft yarn causes the returning (after crimping to allow its insertion into the catheter) of the structure to its initial shape, and particularly tubular to enable said stent graft to fulfill the function which is assigned thereto, and more particularly allow the passage of the blood flow and as a corollary resolve the aneurysm.

The principle of the method of forming the stent graft of the invention has been shown in relation with FIG. 5. Basically, a 3D structure is generated by means of a Rachel loom or double needle bed hook loom (20) (for which, for the simplification of the understanding, the yarn supply modules have not been shown).

Around this double needle bed loom, there rotates (arrow G) a coil (21) of supply of weft yarn (22) made of nickel-titanium alloy having previously undergone a thermal treatment for the optimization of its thermomechanical behavior, assembled on a support (23). Thereby, the weft yarn thus weaves at the level of the needles (24) and of the guides (25) assembled on the needle beds of the RACHEL loom along the forming of the 3D structure at this level.

For this purpose, the weft yarn supply coil (21) is arranged in a plane inscribing perpendicularly to that receiving the double needle beds and passing at the level of the area of cooperation of the needles and of the guides of said needle beds.

The rotating motion of the coil around the two needle beds is obtained by any means, and in any case, by a mechanism synchronized with the cycle of forming of the stitches of the 3D structure on the double needle bed loom. This coil delivers the weft yarn after the passage by a braking device (26) to ensure a correct tension of the weft yarn. This braking is performed either directly on the weft yarn, or on the actual coil. Such braking systems are known per se. They may in particular be of mechanical, electrical, or even electromagnetic nature.

The programming of the pitch of the helix followed by the weft yarn with respect to the production direction of the 3D structure may be directly managed concomitantly with the program of knitting of the 3D background structure. Typically, this programming is such that a permeability close to 0.1 ml/cm²/min, in all cases in accordance with the standard applicable for said determined 3D structure, and particularly capable of fulfilling its tightness function most appropriate to contain the blood flow intended to transit therewithin, is available after impregnation or coating.

The diameter of the tube resulting from the 3D structure is typically 20 millimeters. However, this diameter is likely to vary between 5 and 40 millimeters, according to the patients and to the areas of application of the stent graft, its use being besides not limited to aortic aneurysms only, but also in other vascular pathologies, particularly when a substitution or an inner reinforcement appears to be necessary.

Further, the pitch of the helix of the weft yarn is determined so that said weft yarn turns around the tubular textile structure once for every component mesh, or every 2 to 10 meshes thereof (typically with a mesh density in the order of from 7 to 20 meshes/cm). The structure thus formed enables to ensure an optimal thermomechanical behavior and to avoid risks of plications, endoleaks, and migrations once the stent graft has been introduced within the pathological arterial portion.

The double needle bed has thus been schematically shown in top view in FIG. 6. The front (F1) and rear (F2) needle beds, at the level of which the knitting yarns (27) of the 3D support structure appear, have thus been materialized, as well the schematic layout of the weft yarn inserted by means of a device, such as illustrated in FIG. 7.

Such a device typically comprises a coil (21) for supplying the weft yarn (22), assembled on a circular ring (28). This circular ring is thus assembled around the double needle bed loom. It is rotated for example by means of toothed gears (29), rotated by electric motors (not shown). Such toothed gears mesh on the toothed peripheral edge (30) of said ring. A belt drive system or any other drive means may be used to ensure the rotation. The management of the electric motor(s) actuating the toothed gears is synchronized with the operating cycle of the double needle bed loom, to introduce the weft yarn at the right time at the level of each of the needle beds.

Thus, the weft yarn performs a revolution, and in the example rotates once, around the needle beds in the area when the 3D textile structure obtained by the action of the knitting members assembled on needle beds, respectively needles and guides, is formed. The guides are themselves moved on support bars (31), according to the retained yarn binding program.

The braking device (32) positioned at the coil output, to regulate the tension of the weft yarn, as also been shown in this drawing.

According to the invention and after the forming of the stent graft, a full bath coating thereof with a solution, for example, of collagen, is performed. This optimizes the permeability level measured according to the ISO 25539-2 standard applicable for the stent graft, which turns out being sufficient and meeting the requirements in force. This coating may also be performed by other available techniques, such as for example by sputtering.

The value of the stent graft of the invention is thus obvious. First, its biomimetic character, resulting, on the one hand, from its homogeneous structure and from its embodiment, suppressing any notion of exoskeleton and of suture, and on the other hand, from the mesh pattern of the 3D knit structure, should be underlined. Thereby, the durability of its implantation is favored since risks of in-vivo migration are limited, and its corollary, the decrease of risks of rejection. Second, risks of occlusion are avoided whatever the tortuosity of the arteries that the stent graft is likely to encounter to conform to the specific anatomy of certain blood vessels. Finally, any risk of leak, particularly due to the mesh structure of the 3D structure, is avoided.

1. A vascular stent graft formed of a tubular knitted textile structure, integrating within meshes of said knitted textile structure at least one helical continuous weft yarn extending all along a major dimension of the stent graft, said at least one yarn being made of a shape memory alloy having previously been submitted to a thermomechanical treatment, such that, at the human body temperature, said yarn gives the structure its tubular shape by superelasticity or shape memory effect.

2. A vascular stent graft according to claim 1, wherein the knitted textile structure is obtained on a double needle bed Rachel loom or on a hook loom, according to a weave selected from the Charmeuse, double knit, or weft chain group.

3. A vascular stent graft according to claim 1, wherein the diameter of the knitted textile structure is in the range from 5 to 40 millimeters.

4. A vascular stent graft according to claim 1, wherein said graft comprises a limb obtained after reduction of the diameter of the knitted textile structure, said limb being configured to form a secondary stent graft.

5. A vascular stent graft according to claim 1, wherein the knitted textile structure is made of a biocompatible material selected from the group comprising PET (polyethylene terephthalate), polypropylene, ePTFE, and biodegradable or bioresorbable materials of PGA type.

6. A vascular stent graft according to claim 1, wherein the weft yarn made of a shape memory material is made of nickel-titanium, and wherein said yarn is continuously inserted into the meshes of said knitted textile structure.

7. A vascular stent graft according to claim 1, wherein the weft yarn has a diameter in the range from 50 to 200 micrometers.

8. A vascular stent graft according to claim 1, wherein a longitudinal deformation of the stent graft, that is, along its major dimension, is in the range from 0 to 30%.

9. A vascular stent graft according to claim 1, wherein said graft is coated with collagen, albumin, or gelatin, to provide said stent graft with a permeability close to 0.1 ml/cm²/min regarding the viscosity of the blood flow, and to avoid leaks while respecting the biocompatibility of the stent graft.

10. A vascular stent graft according to claim 9, wherein the collagen, albumin, or gelatin coating also comprises heparin, carbon, or a fluoropolymer.

11. A method of forming a vascular stent graft, comprising:
forming by warp stitch knitting on a double needle bed loom two textile layers joined together at the level of edges of said layers oriented in a production direction of said loom to later define a tubular structure;
and inserting a continuous weft made of a shape memory yarn at the level of the double needle bed, inserting into the meshes on said two layers with an offset in the production direction, the continuous weft being conveyed to the level of the needle beds by revolution around said beds, typically helically with respect to the production direction of the loom.

12. A method of forming a vascular stent graft according to claim 11, wherein a pitch of the helix formed by the shape memory yarn is constant and 1/1, that is, the weft yarn makes turns around the tubular textile structure once for every stitch.

13. A method of forming a vascular stent graft according to claim 11, wherein a pitch of the helix formed by the shape memory yarn is constant and is in the range from ½ to 1/10.

14. A method of forming a vascular stent graft according to claim 11, wherein the double needle bed loom is a RACHEL loom or a hook loom, wherein the weft yarn is inserted at the level of the double needle bed of said loom with an offset of this insertion in the production direction, this continuous weft being conveyed to the level of the two needle beds by revolution around said needle beds and thus concomitantly to the forming of the two textile layers resulting from the knitting at the level of the two needle beds.

15. A method of forming a vascular stent graft according to claim 14, wherein the weft yarn undergoes a previous corrugation before its being inserted into the double needle bed of the RACHEL loom or of the hook loom.
